(19) United States
(12) Patent Application Publication
(10) Pub. No.: US 2016/0346560 A1
(43) Pub. Date: Dec. 1, 2016

(54) Diathermy Heat Applicator Array with Cancellation of Extraneous Incidental Radiation

(71) Applicant: Regear Life Sciences Inc., Pittsbrugh, PA (US)

(72) Inventors: Robert M. Unetich, Pittsburgh, PA (US); James A. Eastburn, Pittsburgh, PA (US); Michael J. Thomas, Severna Park, MD (US)

(21) Appl. No.: 15/165,597

(22) Filed: May 26, 2016

Related U.S. Application Data
(60) Provisional application No. 62/166,306, filed on May 26, 2015.

(51) Int. Cl. A61N 1/40

(52) U.S. Cl. CPC A61N 1/403

(57) ABSTRACT

The present invention pertains to the field of medical diathermy and addresses issues of incidental undesired radiation in the prior art. The invention uses a multitude of pairs of intentional radiators with each pair driven with an identical but out-of-phase signal. The pairs or radiators are spaced near the target tissue in close proximity to the target but sufficiently distant from each other so as not to cancel the nearby tissue heating effects. Local heating of tissue is achieved by the high electric and magnetic field intensities coupling and inducing electric currents in conductive tissue.

### BACKGROUND

The desirable effects of the deep heating of human tissue with radio frequency energy, known as diathermy, is accomplished by generating the required level of electromagnetic energy and radiating it towards the target tissue. Due to the long wavelengths usually involved, with wavelengths normally in the tens of meters, it is not possible to focus all of the energy at the target without also radiating energy in other directions. This can cause heating of other conductors, including human tissue and it can cause interference affecting other nearby electronic equipment. For example, U.S. Pat. No. 4,527,550 describes a diathermy system limited to use in a shielded room.

Numerous means have been employed to minimize this incidental undesired radiation including various shielding methods for example U.S. Pat. No. 4,305,115 describes an Electrostatic Shield for use in shortwave diathermy and an improved shield is described in U.S. Pat. No. 8,489,201, but the effectiveness of shielding depends greatly on the density and conductivity of the shielding and the power absorbed by shielding is lost as heat, reducing efficiency. This necessitates the use of higher power generators capable of supplying sufficient heat to target tissue while dissipating the remaining power in some manner as heat and residual incidental radiation.

### SUMMARY

The present invention addresses deficiencies in the prior art, such as the above-mentioned, by using a multitude of pairs of intentional radiators with each pair driven with an identical but out-of-phase signal. The pairs or radiators are spaced near the target tissue in close proximity to the target but sufficiently distant from each other as to not cancel the nearby tissue heating effects. This causes local heating of tissue because of the high electric and magnetic field intensities coupling and inducing electric currents in conductive tissue. At any distance away from the radiator, that is large compared to the spacing to the target, the out-of-phase fields substantially cancel, greatly reducing the likelihood of interference or undesired tissue heating far away from the target.

In accordance with the present invention, the methods as outlined above, in combination with shielding techniques using localized wiring provide a beneficial reduction, potentially exceeding two orders of magnitude, of unwanted fields. This dramatic reduction in extraneous radiation provides the ability to conduct diathermy heat treatment at locations previously deemed unacceptable, such as the domestic environment, which can be susceptible to interference to other electronics in the home.

### BRIEF DESCRIPTION OF THE DRAWINGS

FIG. 1 An example of splitter and phase shifter embodiment, with simple transformer

FIG. 2. Turns-ratio of transformer selected to achieve the proper distribution of voltages to each heat applicator.

FIG. 3 Flat printed circuits, with connecting coaxial cables.

FIG. 4 Alternate embodiment of a coil design using wire instead of printed coils.

FIG. 5 An embodiment, a system with four diathermy radiators.

FIG. 6 Radiating pairs assembled with necessary splitters to achieve effective radiation of target tissue while canceling far field radiation effects

FIG. 7 Fabric carrier and wire shield mounted thereon, for reduction of quadrature field.

FIG. 8 Wilkinson Splitter

### GLOSSARY

For purposes of this document, “far field” is used to mean more than a few wavelengths away from a source of radiation.

### DETAILED DESCRIPTION

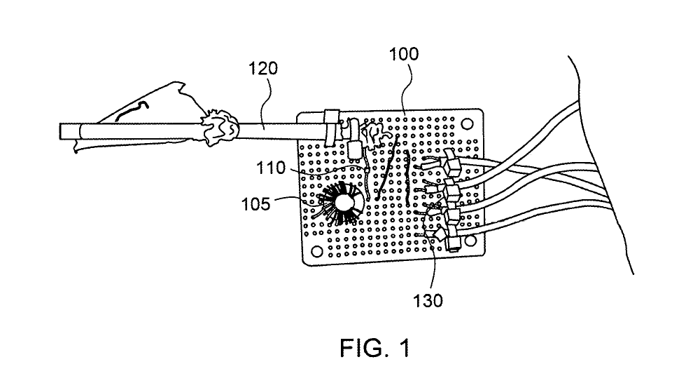

In a preferred embodiments of the present invention, a single source signal delivers its signal via shielded cable to a power splitter and phase inverter. The splitter and phase shifter 100 can take numerous forms. A suitable splitter and phase shifter is a simple transformer 105 an example of which, having 4 secondary windings 130, is shown in FIG. 1. The primary 110 of this transformer is connected to the source signal 120.

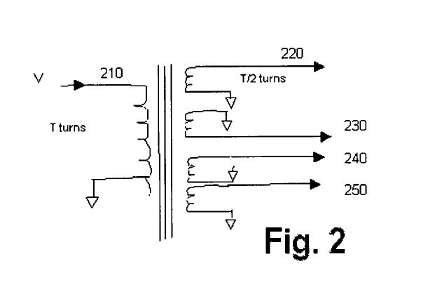

The transformer has multiple secondary windings and any individual secondary winding can be used to deliver a voltage to a desired heat radiator. Another secondary can easily deliver an out-of-phase identical amplitude signal to a second heat applicator by reversing the output connections of that secondary. The turns-ratio of said transformer can selected to achieve the proper distribution of voltages to each heat applicator, as seen in FIG. 2. FIG. 2 shows a 4 way power splitter and phase shifter with a primary of T turns and four secondary windings, each with T/2 turns and each producing ½ V output voltage. Shown are input voltage 210, output #1 zero phase reference 220, output #2 −180 degree phase shift 230, output #3 zero degrees phase shift 240, output #4 −180 Degrees phase shift 250.

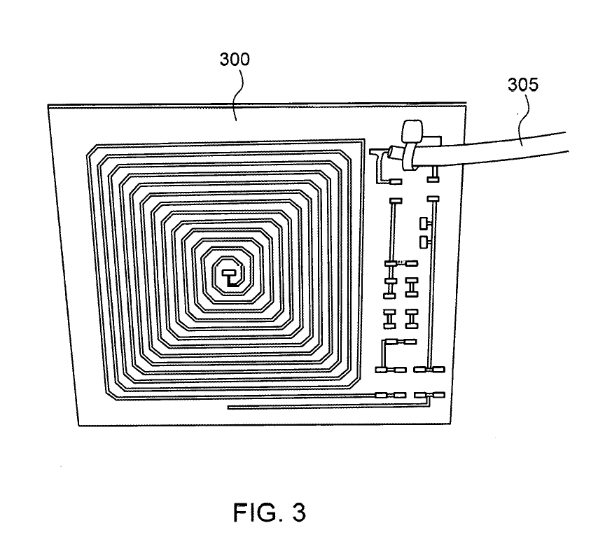

In an embodiment of the invention with two diathermy radiators, a transformer splitter has two secondaries connected out-of-phase to two resonant coils. In embodiments, these coils are flat printed circuits 300, with connecting coaxial cables 305, as seen in FIG. 3. For clarity, tuning components are not shown.

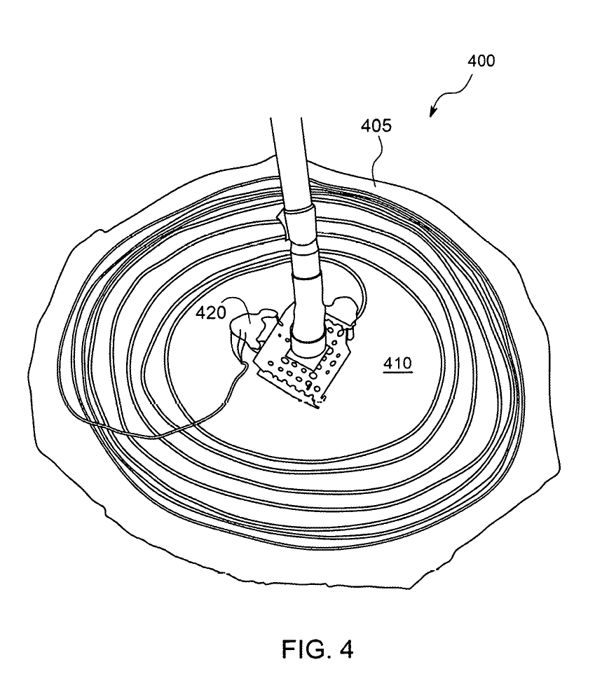

An alternate embodiment of a coil design 400 using wire 405 instead of printed coils is shown in FIG. 4. In this instance, high resistivity wire 410 is wound to achieve the required inductance and resistance simultaneously, although the manufacturability may be degraded by the need for an exact inductance from a hand wound coil of wire. Tuning components 420 are also shown.

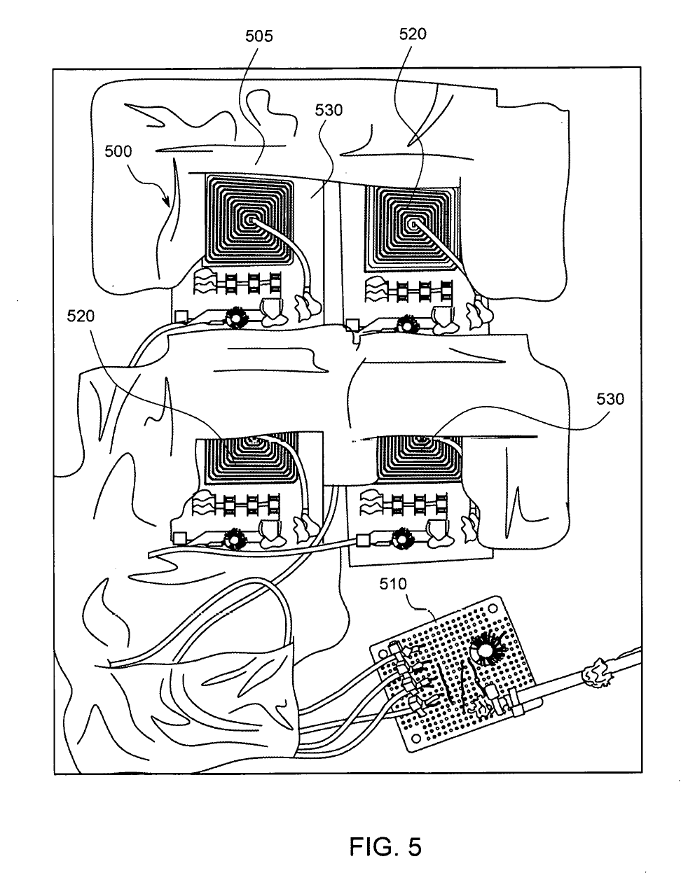

In an embodiment, a system 500 with transformer and four diathermy radiators 505, as shown in FIG. 5, the splitter 510 drives two radiators 520 with an in-phase signal and two with an out-of-phase signal 530. The array is arranged in a checkerboard fashion and the heat applicators are each placed near the target tissue.

In the embodiments as seen above in FIGS. 4 & 5, designed to preferably operate at 13.56 MHz, a common diathermy frequency of choice, the heat applicators are resonant spiral inductors of approximately ten centimeters in diameter. Tissue placed nearby, that is, within one centimeter of a spiral at location 1, receives intense radiation and this causes heating.

At another heat radiator, location 2, spaced for instance at 10 cm. or greater away, the fields caused at location 2 by radiator number 1 will be small and little cancellation or enhancement of the fields will occur.

At distances further away from the array, for instance at 100 cm, the radiation effects will add vectorially. If two such sources are in-phase and two sources are out-of-phase, significant cancellation will occur.

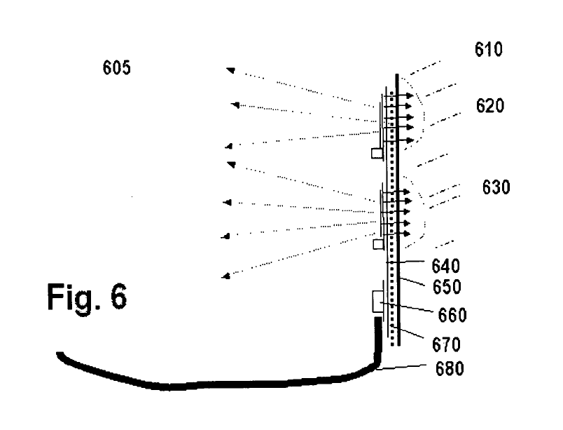

A diagram, FIG. 6, will illustrate examples of embodiments. Any combination of radiating pairs is assembled with the necessary splitters to achieve effective radiation of target tissue while canceling far field radiation effects using the techniques described above. This diagram of a heat applicator (with radiation indicated by arrows) shows non-tissue side 605, having incidental undesired radiation that cancels at distances removed from the out-of-phase radiators. Also shown are tissue side of skin 610, approximate area heated 620 depicted as a curve, the out-of-phase radiator 630, the radiator carrier assembly 640, target human skin 650, the transformer 660, (connections to radiators not shown) the thin foam spacer/fabric 670 and the input cable to generator 680.

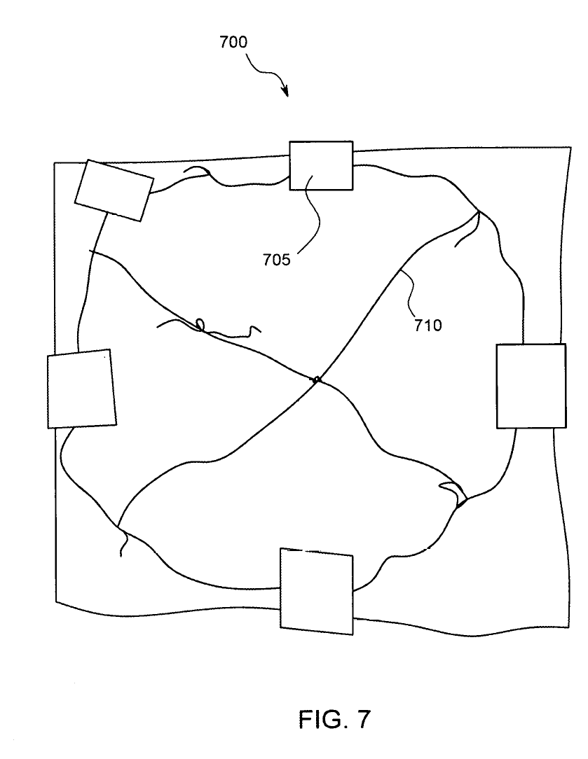

In a further embodiment, the resonant coil pairs are installed in a suitable fabric carrier, for example as shown in FIG. 7. In this embodiment 700, two pairs (not shown), each with its own splitter, are inserted in a carrier. Each pair is then driven by a generator and splitter designed to deliver the desired power with the proper phase relationships. It may be preferable to have a flat fabric carrier 705 driven by a suitable splitter with four outputs, achieving the checkerboard pattern desired to maximize far field cancellation.

Further shown in FIG. 7, to further reduce quadrature radiating electric fields, is the combination of the methods as outlined above with shielding techniques using localized thin cross wiring 710 mounted on the fabric carrier.

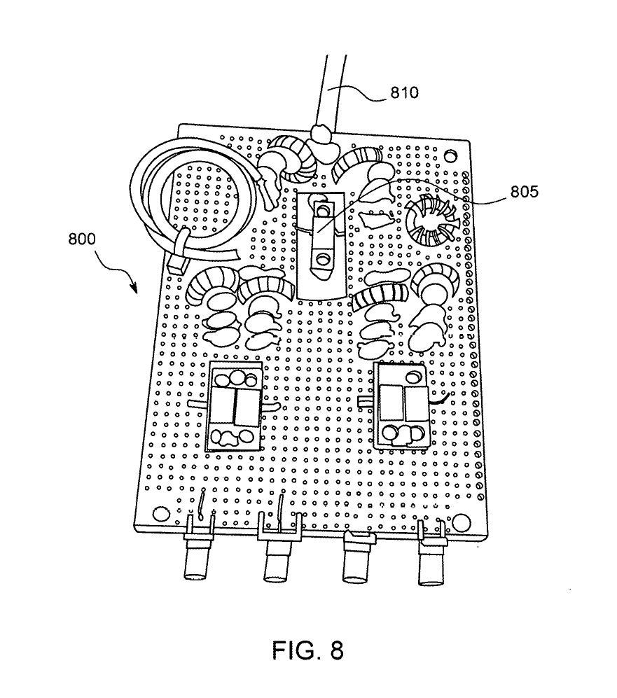

FIG. 8 shows, in yet a further embodiment 800, the use of a Wilkinson Splitter 805 with phase delay provided by a phasing coil 810.

Those experienced in the field of this invention should, based on the detailed descriptions of the objectives and new methods, be able to understand the logical possible variations. They will be able to adopt appropriate strategies depending on the various applications and needs of diathermy applicators, not specifically shown in this application, but within the general goals and objectives of this invention.

Examples disclosed are intended to be limiting only as reflected in the appended claims.

I claim:

1. A diathermy applicator for treatment, in a treatment area, of a patient's designated target tissue comprising at least one pair of radiators connected to driving currents, wherein;
said driving currents are substantially identical except for a phase separation of degree sufficient to diminish harmful radiation reaching persons or apparatus housed in said treatment area
said at least one pair is spaced near the target tissue in close proximity to the target sufficiently distant from each other as to not cancel the nearby tissue heating effects.

2. The diathermy applicator of claim 1 wherein said phase separation is created by a splitter via shielded cable to a power splitter and phase inverter.

3. The diathermy applicator of claim 1 wherein said radiation is in the range of 4-40 MHz

4. The diathermy applicator of claim 2 wherein said splitter and phase shifter comprise a simple transformer having an even number of at least 4 secondary windings.

5. The diathermy applicator of claim 2 further comprising a second pair of radiators and splitters, substantially identical to the first said pair, wherein said two pairs are arranged in checkerboard fashion having arrays and columns, one said pair driven with an in-phase signal and the other with an out-of-phase signal in such a way that array- and column-wise adjacent radiators are out of phase.

6. The diathermy applicator of claim 5 further comprising a plurality of said radiating pairs and splitters.

7. The diathermy applicator of claim 1 wherein said phase separation is approximately 180 degrees.

8. The diathermy applicator of claim 3 wherein said radiation is in the range of 7-23 MHz

9. The diathermy applicator of claim 8 wherein said radiation is in the range of 9.5-17 MHz

10. The diathermy applicator of claim 9 wherein said radiation is approximately 13.56 MHz.

11. The diathermy applicator of claim 4 further comprising a 4 way power splitter and phase shifter with a primary of T turns and four secondary windings, each with T/2 turns and each producing ½ V output voltage.

12. The transformer splitter of claim 3 wherein said coils comprise flat printed circuits with connecting coaxial cables.

13. The diathermy applicator of claim 3 further comprising a fabric carrier.

14. The diathermy applicator of claim 13 wherein said fabric carrier further a wire shield, for reduction of quadrature field.

15. The diathermy applicator of claim 3 wherein said splitter consists of a Wilkinson Splitter.

16. The transformer splitter of claim 11 having two secondaries, each connected out-of-phase to two resonant coils.
